(19) United States
(12) Patent Application Publication
(10) Pub. No.: US 2004/0235935 A1
(43) Pub. Date: Nov. 25, 2004

(54) Oral pharmaceutical composition containing a statin derivative

(76) Inventors: Francis Vanderbist, Beersel (BE); Antonio Sereno, Melsbroek (BE); Philippe Baudier, Uccle (BE)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/480,131

(22) PCT Filed: Jun. 11, 2002
(86) PCT No.: PCT/BE02/00095

(30) Foreign Application Priority Data
Jun. 12, 2001 (BE) 0100098

(51) Int. Cl. A61K 31/401; A61K 31/366; A61K 31/225; A61K 9/14

(52) U.S. Cl. 514/423; 514/460; 514/548; 424/489

(57) ABSTRACT

Oral stable liquid pharmaceutical compositions comprising at least a statin derivative suspended in at least an oily excipient. The said composition allows to obtain a high bioavailability of the drug after oral administration to mammals. A process for manufacturing the said compositions.

Hypercholesterolaemia plays a crucial role in the development of arteriosclerosis diseases in general and coronary heart disease in particular. The risk of progression of the arteriosclerosis process to coronary heart diseases increases progressively with increasing levels of total serum cholesterol or low-density lipoproteins (LDL) cholesterol at both the individual and the population level.

The statins are reversible inhibitors of the microsomal enzyme HMG-CoA reductase, which converts HMG-CoA to mevalonate. This is an early rate-limiting step in cholesterol biosynthesis. In the present document the term “statin” and the term “HMG-CoA reductase inhibitor” are synonyms and interchangeable since both terms describe the same active ingredients. Inhibition of HMG-CoA reductase by statins decreases intracellular cholesterol biosynthesis, which then leads to transcriptionally upregulated production of microsomal HMG-CoA reductase at cell surface LDL receptors. Subsequently, additional cholesterol is provided to the cell by de novo synthesis and by receptor-mediated uptake of LDL-cholesterol from the blood. This resets intracellular cholesterol homeostasis in extrahepatic tissues, but has little effect on the overall cholesterol balance (Clin. Pharmacokinet. 1997, May, 32(5), 403-425).

The main statins currently used in therapeutics are: pravastatin, simvastatin, lovastatin, fluvastatin, atorvastatin and cerivastatin. Lovastatin, simvastatin and pravastatin are derived from fungi (14, 15).

Simvastatin is a clinically modified 2,2-dimethyl-butyrate analogue of lovastatin. Pravastatin is a purified active metabolite of mevastatin with an open hydroxyacid instead of a lactose ring.

Fluvastatin and atorvastatin have an entirely synthetic origin with a structure distinct from that of the other agents (117).

The attached FIG. 1 shows the chemical structure of some statin molecules

The different statin molecules have widely different physico-chemical properties (Table 1). For instance, their solubility in water is widely different as shown by their partition coefficients (logarithm of partition ratio between octanol and water at pH 7.0), which range from −0.23 to 4.7 (21).

TABLE 1
Physiochemical properties of the HMG-CoA reductase inhibitors fluvastatin,
lovastatin, pravastatin and simvastatin

| | | Lovastatin | | Pravastatin | | Simvastatin | |
|---|---|---|---|---|---|---|---|
| | | | Hydroxy | | Hydroxy | | Hydroxy |
| Property | Fluvastatin | Lactone | acid | Lactone | acid | Lactone | acid |
| M (g/mol) | 416 | 405 | 405 | 447 | 447 | 419 | 419 |
| pKa | 5.5 | | 5.5 | | 5.5 | | 5.5 |
| | (acid) | | | | | | |
| D | 1.5 | 4.3 | 1.7 | −0.23 | | 4.7 | 2.1 |
| Solubility in | 2 | 0.0013 | | 0.18 | 300 | 0.0014 | |
| water | | | | | | | |
| (g/L)^{a} | | | | | | | |
| Vₛₒₗ (ml) | 10 | 15 385 | | 111 | 0.07 | 14 286 | |

^{a}At pH 5, except for fluvastatin at pH 6.
Abbreviations and symbols:
D = partition coefficient (logarithm of partition ratio between octanol and water at pH 7);
M = molecular weight;
Vₛₒₗ = volume of water require to completely dissolve a 20 mg dose at the solubility given in the table.

Those values mean that lovastatin and simvastatin are lipophilic and dissolve about 40,000 to 50,000 times more readily in octanol than in water. Pravastatin dissolves twice as readily in water, whereas fluvastatin and the hydroxyacid forms of lovastatin and simvastatin dissolve 32, 50 and 126 times more readily in octanol, respectively.

All the active compounds are acid, and have pKa values of approximately 5.5.

As summarized in table 2, the pharmacokinetic profiles of the statins are also widely different, but the different statins present the common properties of having a large intra and interindividual variability when they are taken by the oral route.

TABLE 2
Intestinal absorption and systemic availability in healthy
humans (unless otherwise stated) of HMG-CoA reductase
inhibitors fluvastatin, lovastatin, pravastatin and simvastatin

| | | Lovastatin | | |
|---|---|---|---|---|
| | | (hydroxy- | | Simvastatin |
| Property | Fluvastatin | acid) | Pravastatin | (hydroxy acid) |
| Oral dose (mg) | 20 | 20 | 20 | 40 |
| AUC (μg · h/L) | 167 | 15.9 | 90.2 | 58.1 |
| Cₘₐₓ (μg/L) | 181 | 3.1 | 38.4 | 58.1 |
| tₘₐₓ (h) | 0.5-1.5 | 2.8 | 1.1 | 1.2 |
| fₐ (%) | >90 | ˜31 | ˜34 | 60-80^{a} |
| P_{eff} | 2.4 | | | |
| (×10⁻⁴ cm/sec) | | | | |
| F (%) | 10-35 | | 18 | |
| E_{H} (%) | 67 | 62^{a} | 45 | 80^{a} |

^{a}From dogs
Abbreviations and symbols:
AUC = area under plasma concentration-time curve;
Cₘₐₓ = maximum plasma drug concentration;
E_{H} = liver extraction ratio;
F = bioavailability;
fₐ = fraction of the administered dose absorbed;
P_{eff} = effective membrane permeability;
tₘₐₓ = time to Cₘₐₓ.

As said hereinabove, HMG CoA reductase inhibitors have very different physicochemical characteristics.

Chemically, HMG CoA reductase inhibitors can be ranged in two categories: the first category involves the molecules containing a lactose function (lovastatin, simvastatin). The molecules are inactive and need to be metabolized by esterase in the hydroxy-acid form to be active (prodrug). The second category contains molecules available under the form of the hydroxyacid (or salt). This category involves among others pravastatin sodium and fluvastatin sodium.

All the HMG CoA reductase inhibitors are relatively unstable but the molecules containing a lactose function appear particularly unstable. Indeed, the lactose function is very easily hydrolysed and this reaction is still catalyzed by several parameters like oxygen, humidity, acidity, alkalinity and temperature. Therefore it would be of particular interest to dispose of a stable form of HMG CoA reductase inhibitors, easy to manufacture and presenting a good bioavailability of the drug

Consequently the main challenges for formulating a pharmaceutical composition comprising a statin derivative is to obtain a stable pharmaceutical composition with a high bioavailability after oral administration to humans.

### STATE OF THE ART

Little information is published about the efforts made in terms of pharmaceutical formulation on statin molecules.

A number of patents have nevertheless been granted.

The U.S. Pat. No. 5,180,589 describes a pharmaceutical composition which has excellent stability, when dispersed in water has a pH of at least about 9, and includes a drug which is sensitive to a low pH environment such as pravastatin, one or more fillers such as lactose and/or microcrystalline cellulose, one or more binders, such as microcrystalline cellulose or polyvinylpyrrolidone, one or more disintegrating agent such as croscarmellose sodium, one or more lubricant such as magnesium stearate and one or more basifying agent such as magnesium oxide.

The U.S. Pat. No. 5,356,896 describes a pharmaceutical dosage form comprising an HMG-CoA reductase inhibitor compound, e.g. fluvastatin sodium, which is stabilized against pH-related degradation by an alkaline stabilizing medium capable of maintaining at pH of at least 8 to an aqueous solution or dispersion of the composition.

The U.S. Pat. No. 6,235,311 describes a pharmaceutical composition which is useful for cholesterol lowering and reducing the risk of myocardial infraction, which includes a statin, such as pravastatin, lovastatin, simvastatin, or atorvastatin, cerivastatin or fluvastatin, in combination with aspirin, in a manner to minimize interaction of aspirin with the statin and minimize side effects of aspirin. A method for lowering cholesterol and reducing risk of a myocardial infraction employing such composition is also provided.

The U.S. Pat. No. 5,225,202 describes an enteric coated pharmaceutical composition which includes a medicament which is sensitive to a low pH environment of less than 3, such as pravastatin, which composition is preferably in the form of pellets which include an enteric coating formed of neutralized hydroxypropylmethylcellulose phthalate, plasticizer and anti-adherent. The so coated pellets have good resistance to deterioration at pH less than 3 but have good drug release properties at greater than 3.

The WO 00/76482 patent describes a pharmaceutical form with increased bioavailability obtained by solubilizing a lipid-regulating agent in one or more components, then solidifying it by adding one or more solid or semi-solid constituents.

The main problem met with this kind of formulations is that heating the formulation to at least 50° C. is required, what can provoke a significant degradation of the statin derivatives.

Furthermore, the amount of excipients needed for dissolving the active ingredient is very important, what makes the final pharmaceutical form very big and consequently very difficult to administer to humans.

The WO 00/57918 patent is directed to a formulation comprising a lipid-regulating agent dissolved in a mixture of an oil and one or more surfactants. The concentrate forms a fine emulsion when water is added in the composition.

The main disadvantage of the invention hereinabove is that the presence of water in a composition is often incompatible with the capsule in which the formulation must by filled to form the final pharmaceutical form. On the other hand, the amount (or volume of excipients) needed to provide the adequate formulation is very important and makes the pharmaceutical form particularly difficult to administer to patients.

The WO 00/57859 patent is directed to a formulation comprising a lipid-regulating agent in at least one oil and one emulsifier blend, then the resulting mixture is transformed in an emulsion upon dilution in an aqueous medium.

The main disadvantage of this invention is again the compatibility of water with the capsule shell and/or the other excipients and/or active ingredients. For instance, the statin derivatives possessing a lactose group will be very rapidly hydrolyzed in the presence of water.

Furthermore, the quality of excipients needed to obtain the final pharmaceutical form does not allow the manufacturing of a practical, easy-to-swallow form for the patient.

Indeed, enormous amounts (or volumes) of excipients are needed to make the final pharmaceutical form.

### SUMMARY OF THE INVENTION

It is an object of the present invention to provide a stable pharmaceutical composition containing a statin derivative. It is also an object of the present invention to provide a composition presenting a high bioavailability of the drug and/or its active metabolite(s) after oral administration to humans. It is another object of the present invention to provide a safe, cheap, ecological rapid and easy process to manufacture the said composition.

### BRIEF DESCRIPTION OF THE DRAWINGS

FIG. 1 gives the Chemical structure of some HMG-CoA reductase inhibitor compounds.

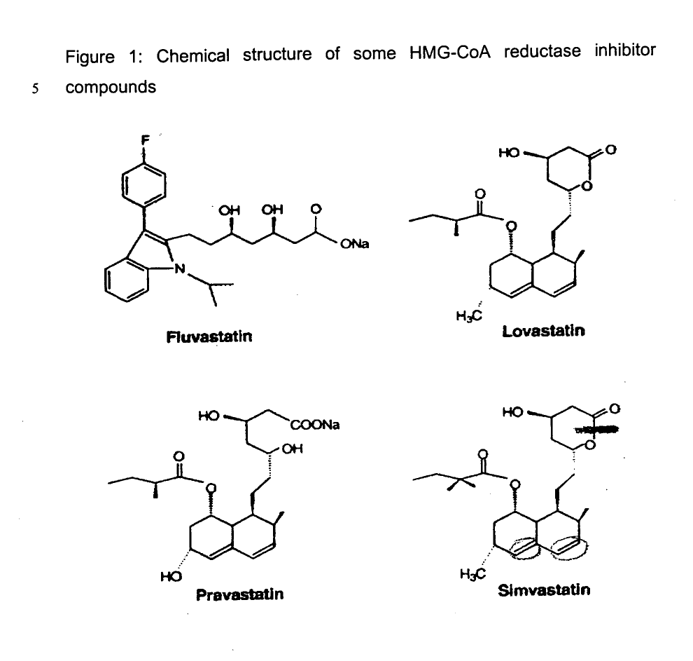

### BACKGROUND OF THE INVENTION

It was discovered that liquid oily formulations could be an interesting alternative for the formulation of HMG CoA reductase inhibitors both in term of stability of the drug in the final composition and in term of the bioavailability of the drug in humans.

It is also a purpose of the present invention to describe a process for manufacturing a pharmaceutical composition, which process is ecological, economically advantageous, rapid, simple and safe for the operators.

The present invention relates to a liquid oily pharmaceutical composition containing at least one HMG-CoA reductase inhibitor agent.

The liquid suspension contains advantageously at least one oily excipient and one stabilizing agent e.g. one or more antioxidant or preservative agent(s) or combination of both preservative and antioxidant agents.

The oral pharmaceutical formulation of the invention comprises at least one pharmaceutically acceptable carrier and a water free suspension of solid particles containing at least one statin or statin derivative with a particle size of less than 500 μm (advantageously lower than 200 μm) in at least one pharmaceutical oil or mix of pharmaceutically acceptable oils. The oil or mix of oils is liquid at room temperature (20° C.), preferably at temperature below 20° C., such as temperature below 10° C., or even at temperature below 0° C. The viscosity of the suspension is advantageously controlled so that said viscosity is lower than 100 cp (centipoises) at 20° C., advantageously lower than 50 cp at 20° C.

The pharmaceutically acceptable carrier is adapted for releasing said water free suspension in the gastric tract and/or in the intestine tract, especially in the intestine tract and/or controlling the release of said water free suspension in the gastric tract and/or in the intestine tract, especially in the intestine tract. It means that when a patient is orally administered with a formulation of the invention, the water free oily suspension of solid statin/statin derivative particles is liberated as a suspension in the gastric tract and/or intestine tract. The carrier is for example a capsule containing the suspension, said capsule being dissolved and/or disintegrated and/or destroyed in the gastric and/or intestine tract, whereby ensuring the release of the water free oily suspension in the gastric and/or intestine tract.

The oral formulation of the invention comprises advantageously an amount of statin or statin derivatives corresponding to at least 1,5%, advantageously at least 3%, preferably at least 4% by weight of the total weight of the water free oily suspension, preferably of the oral formulation.

According to preferred embodiment, the solid particles containing at least one statin or statin derivative have a weight average particles size lower than 100 μm, advantageously lower than 50 μm, preferably lower than 50 μm, most preferably comprised between 2 and 30 μm, such as about 10 μm or about 20 μm.

According to a detail of an embodiment, the water free suspension comprises about 0.5 to 50% of at least one statin or statin derivative and about 30% to 99% of a pharmaceutical oil or mix of oils liquid at room temperature.

The statin or statin derivative is for example selected from the group consisting of simvastatin, lovastatin, fluvastatin, atorvastatin, cerivastatin, pravastatin and combinations of several of those molecules.

The pharmaceutical oil or mix of oils is advantageously selected from the group consisting of soyabean oil, olive oil, peanut oil, arachide oil, paraffine, a mono-, di, or triglyceride of C6-C18 fatty acids, polyethyleglycol derivatives, and mixtures of several of those oils

The suspension can further comprise at least one pharmaceutically acceptable excipient selected from the group consisting of antioxidants, stabilizers, viscosifiers, emulsifiers, surfactants and mix of several of those excipients.

The suspension advantageously further comprises at least one pharmaceutically acceptable antioxidant or a combination of antioxidants selected from the group consisting of Vitamine E derivatives, methoxyphenol derivatives such as butylhydroxyanisole, butylhydroxytoluene, propyl gallate and mixtures thereof.

As the oral formulation of the invention is extremely stable, the suspension comprises substantially no degradation product or products of the statin or statin derivatives after 1 month storage at 25° C. in a relative air humidity of 60%, the weight ratio degradation product or products/statin or statin derivative being advantageously lower than 0.5, preferably lower than 0.2 after 1 month storage at 25° C. in a relative air humidity of 60%. For example, the weight ratio degradation product or products/statin or statin derivative is advantageously lower than 0.5, preferably lower than 0.2 after at least 2 months (such after 3 months or even after 6 months) storage at 25° C. in a relative air humidity of 60%.

According to a detail of an embodiment, the suspension is filled into pharmaceutically acceptable capsules.

The oral formulation comprises preferably a pharmaceutical dose of statin or statin derivative suitable for administration to humans.

The suspension comprises preferably an oily phase composed by a mix of soyabean oil and medium chain triglycerides, said mix being advantageously free of emulsifiers and surfactants.

The suspension preferably comprises one or more vitamine E derivative(s) combined with one or more methoxyphenol derivative(s) or with other acceptable antioxydants.

The invention relates also to a manufacturing process for the preparation of an oral formulation of the invention, in which a suspension of statin or statin derivatives in a water free liquid containing one or more pharmaceutically acceptable oils is prepared, and in which the so prepared water free suspension is filled in a carrier.

The process of claim 15, in which a water free and statin free oil liquid is prepared by mixing at least one or more pharmaceutically acceptable oils, with one or more antioxydants (advantageously without emulsifier or surfactant), and in which particles of statin or statin derivatives are mixed with the water free liquid up to the preparation of a homogeneous water free suspension.

The way to discover that water free oily liquid formulations of the invention was the best pharmaceutical form for statin derivatives both in terms of stability and bioavailability has been long and surprising.

Indeed, in a first step, we have formulated a statin derivative (simvastatine) as a tablet form. Two tablets have been formulated, one being a direct compression tablet, (batch: 7B2001/B), the other a tablet made after a wet granulation process (batch: 06D2001).

The tablets manufactured by direct compression (batch 7B2001 B) contained as inactive ingredient : lactose monohydrate, povidone, crospovidone, citric acid and magnesium stearate and the tablets made by wet granulation (batch 06D2001) contained as inactive ingredient: lactose monohydrate, microcrytalline cellullose, povidone, citric acid and magnesium stearate. Both formulations are classic and contain well known and widely described inactive ingredients.

Both tablet formulations have been administered to 6 human volunteers in a single dose, cross-over pharmacokinetic study. The reference formulation was ZOCOR® 40 mg (Merck, Sharp & Dohme). The formulation of the tablets is given hereinbelow: It should be noted that, for a better understanding of the present invention, all the pharmacokinetic data given are normalized to 40 mg for simvastatin. Furthermore, all the pharmacokinetic data are expressed as a percentage obtained by dividing the value obtained with reference drug and the percentage obtained with new drug developed. Consequently, a ratio of 100% described bioequivalent products, a ratio>100% described a bioavailability superior for the reference drug while a ratio<100% described a bioavailability superior for the new drug making the object of the invention

The results of the pharmacokinetic study clearly indicate that the bioavailability of simvastatin after the two formulated tablets was lower than with the reference.

TABLE 3
comparative pharmacokinetic data of simvastatin and its
active metabolite 4-hydroxy simvastatin after oral
administration to 6 healthy volunteers of two tablets
formulations versus Zocor ®

| | | |
|---|---|---|
| Zocor | 7B2001B | 06D2001 |

SIMVASTATIN (ratio reference/test)

| | | | |
|---|---|---|---|
| AUC (0-12 h) (ng · h/ml) | 100 | 110 | 209 |
| Cmax (ng/ml) | 100 | 162 | 301 |

4-HYDROXY-SIMVASTATIN (ratio reference/test)

| | | | |
|---|---|---|---|
| AUC (0-12 h) (ng · h/ml) | 100 | 123 | 163 |
| Cmax (ng/ml) | 100 | 148 | 179 |

It can be observed that both tablets tested present ratio of AUC and Cmax very significantly higher than 100%. That means that the tablets developed present a lower bioavailability of simvastatin and its active metabolite 4-hydroxy simvastatin than the reference

It can be concluded that the formulation and/or manufacturing process of simvastatin has a great influence on its bioavailability after oral administration to humans. It can also be concluded that it is not obvious for a man skilled in the art to obtain a pharmaceutical form presenting a high bioavailability of simvastatin and/or its active metabolite 4-hydroxy simvastatin, since classical formulations give very low results.

Ina second step, it was decided to formulate the statin derivative as a semi-solid form in which the statin would be at least partially dissolved to increase the bioavailability of the drug. Different compositions have been formulated using for instance polyglycolized glycerides (Gelucire®) as solubilizing agents. Different examples of such compositions are given hereinbelow.

TABLE 4
examples of semi-solid formulation of simvastatin

| Ingredients | 25F2001B | 25F2001A | 22H2001 |
|---|---|---|---|
| simvastatin | 40 | 40 | 40 |
| Gelucire 44/14 | 350 | / | / |
| Gelucire 50/13 | / | 583 | 583 |
| Myverol | / | / | / |
| Transcutol | / | / | / |
| Vit E TPGS | 30 | 50 | 50 |
| PEG 600 | / | / | 33 |
| butylhydroxyanisole | 0.04 | 0.04 | 0.04 |
| butylhydroxytoluene | 0.04 | 0.04 | 0.04 |

A pharmacokinetic study was performed on 6 volunteers with the formulations given hereinabove and compared to ZOCOR® 40 mg (Merck, Sharp and Dohme) as reference product, and as it can be observed hereinbelow, the pharmacokinetic parameters had significantly increased for the semi-solid formulation in comparison with the reference product since the ratio {(reference/new formulation)×100} is significantly inferior to 100%.

TABLE 5
comparative pharmacokinetic data of simvastatin and its
active metabolite 4-hydroxy simvastatin after oral
administration to 6 healthy volunteers

| | | |
|---|---|---|
| Zocor | 25F2001B | 22H2001 |

SIMVASTATIN (ratio reference/test)

| | | | |
|---|---|---|---|
| AUC (0-12 h) (ng · h/ml) | 100 | 87 | 63 |
| Cmax (ng/ml) | 100 | 70 | 39 |

4-HYDROXY-SIMVASTATIN (ratio reference/test)

| | | | |
|---|---|---|---|
| AUC (0-12 h) (ng · h/ml) | 100 | 83 | 50 |
| Cmax (ng/ml) | 100 | 90 | 54 |

Unfortunately, it was found out that the stability data on the semi-solid formulations of simvastatin show a significant instability at 25° C./60% relative humidity after 1 month storage. This instability is principally characterized by the apparition of the main degradation product of simvastatin, the 4-hydroxy simvastatin The stability data are given hereinbelow

TABLE 6
stability of semi-solid formulations of simvastatin

| | % 4-OH simvastatin | | |
|---|---|---|---|
| | 25F2001B | 25F2001A | 22H2001 |
| | (55° C.)* | (38° C.) | (40° C.) |
| Initial | 0.14 | 0.17 | 0.15 |
| 1 month 25° C./60% RH | 4.31 | 1.5 | 1.43 |

*temperature at which the composition is heated to allow the capsule filling

This instability may be provoked by the heating of the formulation necessary to make the composition liquid and hence allow the capsule filling.

We decreased the temperature to 38° C. (25F2001A) during the manufacturing process but the instability of simvastatin after 1 month storage at 25° C./60% RH was still significant.

We then decided andthis is the object of the present invention to formulate the statin derivative as a liquid suspension in an oil to assess its stability. Different compositions of simvastatin as such suspension were formulated, containing at least the statin derivative, one oily vehicle and one or more antioxydant and/or stabilizing agents. The formulations were manufactured without heating or in other words at the ambient temperature.

TABLE 7
examples of formulations of statins in oily suspensions

| Ingredients | 1B2002 | 6L2001 | 17A2002 |
|---|---|---|---|
| simvastatin | 40 | 40 | 40 |
| soya bean oil | 527 | 310 | 585 |
| medium chain triglycerides | 317 | / | 325 |
| Vit E acetate | / | / | 17 |
| Vit E TPGS | / | / | 33 |
| PEG 600 | / | 483 | / |
| butylhydroxyanisole | 0.07 | 0.07 | 0.07 |
| butylhydroxytoluene | / | 0.07 | / |
| propylgallate | 0.07 | / | 0.07 |

The stability of those suspensions after 1 month storage at 25° C./60% RH did not show any significant apparition of degradation products.

TABLE 8
stability of semi-solid formulations of simvastatin

| | % 4-OH simvastatin | | |
|---|---|---|---|
| | 17402 | 8D2002 | 06L2001 |
| Initial | 0.011 | 0.022 | 0.015 |
| 1 month 25° C./60% RH | 0.074 | 0.044 | 0.085 |

We then decided to perform a cross-over pharmacokinetic study with those suspensions on 6 healthy volunteers. The reference drug was ZOCOR® 40 mg. Suprisingly enough, while simvastatin was formulated as a suspension (and not an emulsion or a micellar solution), the bioavailability of the drug had increased in comparison with the reference. Indeed, the pharmacokinetic parameters show a high suprabioavailability of the composition described in the present invention when compared to the reference product. This is surprising since simvastatin having a very poor water solubility, it was not expected that a suspension could offer such a high bioavailability. Indeed, for this kind of drugs, the solubility is often the limiting step for the resoprtion of the drug.

TABLE 9
comparative pharmacokinetic data of simvastatin and its
active metabolite 4-hydroxy simvastatin after oral
administration to 6 healthy volunteers

| | | |
|---|---|---|
| Zocor | 17A02 | 6L2002 |

SIMVASTATIN (ratio reference/test)

| | | | |
|---|---|---|---|
| AUC (0-12 h) (ng · h/ml) | 100 | 86 | 96 |
| Cmax (ng/ml) | 100 | 98 | 95 |

4-HYDROXY-SIMVASTATIN (ratio reference/test)

| | | | |
|---|---|---|---|
| AUC (0-12 h) (ng · h/ml) | 100 | 57 | 73 |
| Cmax (ng/ml) | 100 | 56 | 65 |

It can be observed from thet data given hereinabove that the suprabioavailability of the composition making the object of the present invention is more marked on the active metabolite (4-OH-simvastatin) than on the prodrug simvastatin.

The oily liquid composition is typically a suspension. This has for instance be proven by comparing the measurement of the particle size of simvastatin as raw material (measured by laser diffraction) and the particle size of simvastatin in the oily suspension (measured by hot stage microscopy). Both measures give a mean particle size of about 20 μm. This means that the simvastatin is neither dissolved nor emuslified in the oily excipients. Indeed, since the particle size of the raw material is maintained in the final composition, the composition is a suspension. Furthermore, the crystals of simvastatin in the oily suspensions can be observed very easily with an optical microscope.

The oily vehicle suitable to suspend the statin derivative may be but is not limited to soyabean oil, olive oil, peanut oil, arachide oil, paraffine, a mono-, di, or triglyceride of a C6-C18 fatty acid, a polyethyleneglycol derivative or a mix of several of those oils.

Also advantageous for the stability and, in some cases, the bioavailability of the composition is the addition of an antioxidant agent such as, but not limited to, a Tocopherol derivative like α Tocopherol (Vitamin E), α Tocopherol acetate, Vitamin E TPGS or a methylphenol derivative like butylhydroxyanisol (BHA) or butylhydroxytoluene (BHT).

Although some examples of formulations have been given in table 7, some detailed examples are given hereinbelow to clearly illustrate the invention.

### EXAMPLES

1) 585 g of soya bean oil are mixed together with 325 g of medium chain triglycerides in an adequate mixer (for instance a Bi-Agi® 2.5 liters Lleal, Spain) and at ambient temperature. 33 g of vitamine liquid E TPGs are added slowly and under agitation to the oily vehicle. Then 17 g of acetate of tocopherol are added. 0.07 g of butylhydroxyanisole and 0.07 g of propylgallate are added to the mix. The mixing is maintained until complete dissolution of the antioxidants. Finally, 40 g of simvastatin are added to the mix and the mixing is maintained until completed homogeneisation of the simvastatin suspended in the oily vehicle. The oily mix is then filled into hard gelatin capsules and the capsules are sealed.

2) 310 g of soya bean oil are mixed together with 483 g of polyethyleneglycol 600 in an adequate mixer (for instance a Bi-Agi® 2.5 liters Lleal, Spain) and at ambient temperature. 0.07 g of butylhydroxyanisole and 0.07 g of propylgallate are added to the mix. The mixing is maintained until complete dissolution of the antioxydants. Finally, 40 g of simvastatin are added to the mix and the mixing is maintained until completed homogeneisation of the simvastatin suspended in the oily vehicle. The oily mix is then filled into hard gelatin capsules and the capsules are sealed.

3) 400 g of medium chain triglycerides in filled in an adequate mixer (for instance a Bi-Agi® 2.5 liters Lleal, Spain) at ambient temperature. 70 g of vitamine liquid E TPGs are added slowly and under agitation to the oily vehicle. 0.07 g of butylhydroxyanisole and 0.07 g of propylgallate are added to the mix. The mixing is maintained until complete dissolution of the antioxydants. Finally, 40 g of pravastatin Na are added to the mix and the mixing is maintained until completed homogeneisation of the simvastatin suspended in the oily vehicle. The oily mix is then filled into hard gelatin capsules.

4) Detailed example of manufacturing process of the said pharmaceutical composition.

One of the advantages of the invention relates to the easiness of the manufacturing process of the medication and the rapidity and easiness of the pharmaceutical composition.

Mix soya bean oil with Akomed® at ambient temperature in an adequate mixing tank (Lleal tri-agi® or fryma®)

Add the liquid Vit E TPGS®; maintain the mix below 30° C.

Add the antioxidants butylhydroxianisole and butylhydroxitoluene

Maintain the mixing until complete solubilisation of the antioxidant

Add simvastatin and allow homogenization of the drug

Fill into hard gelatin or hypromellose capsule

Seal the capsule (optional)

Package into blister of HDPET bottle

This manufacturing process presents the advantages to be quite short, ecological, simple, safe for the operators and is very easily applicable at an industrial scale. Furthermoren, no heating is required what is very advantangeous for the stability of statins derivatives.

1. An oral pharmaceutical formulation comprising at least one pharmaceutically acceptable carrier and a water free suspension of solid particles containing at least one statin compound with a particle size of less than 500 μm in at least one pharmaceutical oil or mixture thereof, whereby said oil or mixture thereof is liquid at room temperature (20° C.), in which said pharmaceutically acceptable carrier is adapted for releasing said water free suspension in a tract selected from the group consisting of a gastric tract and intestine tract.

2. The oral formulation of claim 1, which comprises an amount at least one of statin compound corresponding to at least 1,5% by weight of the total weight of the water free suspension.

3. The oral formulation of claim 1, wherein the solid particles containing the at least one statin or statin compound have a particle size of less than 200 μm.

4. The oral formulation of claim 1, in which the solid particles containing the at least one statin compound have a weight average particles size lower than 100 μm.

5. The oral formulation of claim 1, wherein the water free suspension comprises about 0.5 to 50% of the at least one statin compound and about 30% to 99% of the at least one pharmaceutical oil or mixture thereof liquid at room temperature.

6. The oral formulation of claim 1, wherein the at least one statin compound is selected from the group consisting of simvastatin, lovastatin, fluvastatin, atorvastatin, cerivastatin, pravastatin and combinations thereof.

7. The oral formulation of claim 1, wherein the at least one pharmaceutical oil or of oils is selected from the group consisting of soybean oil, olive oil, peanut oil, arachide oil, paraffine, a mono-, di, or triglyceride of C6-C18 fatty acids, polyethyleneglycol compounds, and mixtures thereof.

8. The oral formulation of claim 1, wherein the suspension further comprises at least one pharmaceutically acceptable excipient selected from the group consisting of antioxidants, stabilizers, viscosifiers, emulsifiers, surfactants and a mixture thereof.

9. The oral formulation of claim 1, wherein the suspension further comprises at least one pharmaceutically acceptable antioxidant selected from the group consisting of Vitamine E derivatives, methoxyphenol derivatives and a mixture thereof.

10. The oral formulation of claim 1, wherein the suspension comprises substantially no degradation product or products of the at least one statin or statin compound after 1 month storage at 25° C. in a relative air humidity of 60%.

11. The oral formulation of claim 1, wherein the suspension is filled into pharmaceutically acceptable capsules.

12. The oral formulation of claim 1, which comprises a pharmaceutical dose of the at least one statin compound suitable for administration to a human.

13. The oral formulation of claim 1, in which the suspension comprises an oily phase composed by a mix of soybean oil and medium chain triglycerides.

14. The oral formulation of claim 1, wherein the suspension comprises at least one vitamin E derivative combined with at least one compound selected from the group consisting of methoxyphenol derivatives and acceptable antioxidants different from vitamin E derivative.

15. The oral formulation of claim 1, wherein the water free suspension is free of emulsifier.

16. A manufacturing process for the preparation of an oral formulation comprising at least one pharmaceutically acceptable carrier and a water free suspension of solid particles containing at least one statin compound with a particle size of less than 500 μm in at least one pharmaceutical oil or mixture thereof whereby said at least one oil or mixture thereof is liquid at room temperature (20° C.), in which said pharmaceutically acceptable carrier is adapted for releasing said water free suspension in a tract selected from the group consisting of the gastric tract and the intestine tract, wherein the suspension of the at least one statin compound in a water free liquid containing at least one pharmaceutically acceptable oil or mix of oils liquid at room temperature is prepared, and in which the so prepared water free suspension is filled in a carrier.

17. The process of claim 16, wherein the water free and statin free oil liquid is prepared by mixing at least pharmaceutically acceptable oil or mix of oils liquid at room temperature, at least one antioxidant and at least one compound selected from the group consisting of emulsifiers and surfactants, and in which particles of the at least one statin compound are mixed with the water free liquid up to the preparation of a homogeneous water free suspension.

18. The oral formulation of claim 1, which comprises an amount of the at least one statin compound corresponding to at least 3% by weight of the total weight of the water free suspension.

19. The oral formulation of claim 1, which comprises an amount of the at least one statin compound corresponding to at least 4% by weight of the total weight of the water free suspension.

20. The oral formulation of claim 1, which comprises an amount of the at least one statin compound corresponding to at least 1.5% by weight of the total weight of the water free formulation.

21. The oral formulation of claim 1, which comprises an amount of the at least one statin compound corresponding to at least 3% by weight of the total weight of the water free formulation.

22. The oral formulation of claim 1, wherein the solid particles containing the at least one statin compound have a weight average particle size of lower than 50 μm.

23. The oral formulation of claim 1 wherein the solid particles containing the at least one statin compound have a weight average particle size of between 2 and 30 μm.

24. The oral formulation of claim 1, wherein the suspension further comprises at least one pharmaceutically acceptable antioxidant selected from the group consisting of Vitamin E derivatives, methoxyphenol derivatives and mixtures thereof.

25. The oral formulation of claim 1, in which at least one pharmaceutically acceptable antioxidant is selected from the group consisting of butylhydroxyanisole, butylhydroxytoluene, propyl gallate and mixtures thereof.

26. The oral formulation of claim 1, in which the suspension comprises substantially no degradation product or products of the at least one statin compound after 1 month storage at 25° C. in a relative air humidity of 60%, the weight ratio degradation product or products/statin or statin derivative being advantageously lower than 0.5% after 1 month storage at 25° C. in a relative air humidity of 60%.

27. The oral formulation of claim 1, in which the suspension comprises substantially no degradation product or products of the at least one statin compound after 1 month storage at 25° C. in a relative air humidity of 60%, the weight ratio degradation product or products/statin or statin derivative being advantageously lower than 0.2 % after I month storage at 25° C. in a relative air humidity of 60%.

28. The oral formulation of claim 1, in which the water free suspension is free of surfactant.

29. The oral formulation of claim 1, wherein the water-free suspension has a viscosity of lower than 100 cp at 20° C.

30. The oral formulation of claim 29, wherein the water-free suspension has a viscosity of lower than 50 cp at 20° C.

31. A method of reducing formulation of a degradation product of at least one HMG CoA reductase inhibitor in a pharmaceutical composition, which comprises mixing said at least one HMG CoA reductase inhibitor in a water-free suspension comprising at least one pharmaceutically acceptable oil being liquid at room temperature.

32. The method of claim 31, wherein said at least one HMG CoA reductase inhibitor is a statin compound or compounds.

33. The method of claim 32, wherein the statin compound or compounds is selected from the group consisting of simvastatin, lovastatin, fluvastatin, aforvastatin, cerivistatin, provastatin and mixture thereof.

34. The method of claim 33, wherein the statin compound is simvastatin, and the degradation product is 4-hydroxy simvastatin.

35. A method of treating hypercholesterolaemia in a mammal, which comprises orally administering an effective amount of the oral pharmaceutical formulation of claim 1 to a mammal in need thereof.

36. The method of claim 35, wherein said mammal is a human.
